(19) United States
(12) Patent Application Publication
(10) Pub. No.: US 2019/0344102 A1
(43) Pub. Date: Nov. 14, 2019

(54) CPAP Storage and Organizing Unit

(71) Applicant: Anthony Jerome Bradford, Hampton, VA (US)

(72) Inventor: Anthony Jerome Bradford, Hampton, VA (US)

(21) Appl. No.: 15/973,530

(22) Filed: May 8, 2018

(51) Int. Cl. A62B 25/00; A61M 16/06; A61M 16/08; A61M 16/00

(52) U.S. Cl. CPC A62B 25/00; A61M 16/0057; A61M 16/08; A61M 16/06

(57) ABSTRACT

A continuous positive airways pressure machine rack structured and arranged to neatly and conveniently store CPAP accessories, with easy-to-use hose clamps, mask hooks, wire management grommet holes, and accessory shelves.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of organizing devices for medical equipment and more specifically relates to a continuous positive airways pressure machine rack structured and arranged to neatly and conveniently store CPAP accessories, with easy-to-use hose clamps, mask hooks, wire management grommet holes, and accessory shelves.

### 2. DESCRIPTION OF THE RELATED ART

Continuous positive airway pressure (CPAP) is a form of positive airway pressure ventilator, which applies mild air pressure on a continuous basis to keep the airways continuously open in people who are able to breathe spontaneously on their own. It is an alternative to positive end-expiratory pressure (PEEP). Both modalities stent the lungs' alveoli open and thus recruit more of the lung's surface area for ventilation. But while PEEP refers to devices that impose positive pressure only at the end of the exhalation, CPAP devices apply continuous positive airway pressure throughout the breathing cycle. Thus, the ventilator itself does not cycle during CPAP, no additional pressure above the level of CPAP is provided, and patients must initiate all of their breaths.

CPAP typically is used for people who have breathing problems, such as sleep apnea. CPAP also may be used to treat preterm infants whose lungs have not yet fully developed. For example, physicians may use CPAP in infants with respiratory distress syndrome. It is associated with a decrease in the incidence of bronchopulmonary dysplasia. In some preterm infants whose lungs haven't fully developed, CPAP improves survival and decreases the need for steroid treatment for their lungs. In resource-limited settings where CPAP improves respiratory rate and survival in children with primary pulmonary disease, researchers have found that nurses can initiate and manage care with once or twice daily physician rounds.

CPAP therapy utilizes machines specifically designed to deliver a constant flow of pressure. Some CPAP machines have other features as well, such as heated humidifiers. CPAP is the most effective treatment for obstructive sleep apnea, in which the mild pressure from the CPAP prevents the airway from collapsing or becoming blocked. Unfortunately, when using a CPAP machine, tangled mask straps, disconnected nosepieces, and masks falling off the bed or nightstand can keep a user up at night.

Various attempts have been made to solve problems found in racks and stands for medical equipment art. Among these are found in: U.S. Pat. No. 4,892,279 to William G. Lafferty, et al; U.S. Pat. No. 6,224,027 to Linn D. Johnson, et al.; U.S. Pat. No. 5,890,687 to Paul E. Pryor et al.; U.S. Pat. No. 8,020,815 to James Mark Cox; U.S. Pat. No. 5,110,076 to Stephen J. Snyder, et al. This prior art is representative of stands and racks for medical equipment that can be used at home.

Ideally, a continuous positive airways pressure machine rack should be user-friendly and safe in-use and yet should operate reliably and be manufactured at a modest expense. Thus, a need exists for a continuous positive airways pressure machine rack, a continuous positive airways pressure machine rack structured and arranged to neatly and conveniently store CPAP accessories, with easy-to-use hose clamps, mask hooks, wire management grommet holes, and accessory shelves and to avoid the above-mentioned problems.

### BRIEF SUMMARY OF THE INVENTION

In view of the foregoing disadvantages inherent in the known organizing devices for medical equipment art, the present invention provides a continuous positive airways pressure machine rack (Entitled CPAP Storage and Organizing Unit). The general purpose of the present invention, which will be described subsequently in greater detail is to provide a continuous positive airways pressure machine rack structured and arranged to neatly and conveniently store CPAP accessories, with easy-to-use hose clamps, mask hooks, wire management grommet holes, and accessory shelves.

A continuous positive airways pressure machine rack comprising: a bottom panel; a top panel; a back panel including an aperture; a first side panel; a second side panel; a shelf panel including an aperture; a first hook member; a second hook member; and a clamp member. The bottom panel is adapted to retain a base member of a continuous positive airways pressure machine thereon. Wherein the aperture extends through a middle portion thereof and is adapted to allow electrical cords of the continuous positive airways pressure machine to pass therethrough. Wherein the back panel is connected to and extends between edge portions of the bottom panel and the top panel.

Wherein the first side panel is connected to and extends between edge portions of the bottom panel and the top panel at respective first end portions thereof, and is connected to and extends from the back panel at a first end portion thereof. Wherein the second side panel is connected to and extends between edge portions of the bottom panel and the top panel at respective second end portions thereof, and is connected to and extends from the back panel at a second end portion thereof. Wherein the top panel, the bottom panel, the back panel, and the first and second side panels form an interior volume and an open front area. Wherein the aperture extends through a middle portion thereof and is adapted to allow a hose member of said continuous positive airways pressure machine pass therethrough. Wherein the shelf panel is connected to and extends between respective middle portions of the first and second side panels, and is located adjacent the back panel.

Wherein the first hook member is attached to an inner surface of the top panel and extends downwardly therefrom and is adapted to releasably retain a portion of a mask member of the continuous positive airways pressure machine thereon. Wherein said second hook member is attached to an inner surface of the top panel, extends downwardly therefrom, is spaced from the first hook member, and is adapted to releasably retain a portion of the hose of the continuous positive airways pressure machine thereon. Wherein the clamp member is attached to an inner surface of the top panel, extends downwardly therefrom, is spaced from the first and second hook members, and is adapted to releasably retain a portion of the hose of the continuous positive airways pressure machine thereon.

A combination of a continuous positive airways pressure machine and a continuous positive airways pressure machine rack, the combination comprising: a continuous positive airways pressure machine including a base member including a motor member and a water reservoir; an elongated hose member; and a mask member including a face portion and a strap member. Wherein the elongated hose member is releasably attached to the base member. Wherein the face portion is adapted to be placed on a user's face. Wherein said strap member is adjustably connected to said face portion and is adapted to extend around a user's head to releasably hold the mask member to the user's face.

The present invention holds significant improvements and serves as a continuous positive airways pressure machine rack. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, a Continuous Positive Airways Pressure Machine Rack (Entitled CPAP Storage and Organizing Unit), constructed and operative according to the teachings of the present invention.

FIG. 1 shows a perspective view illustrating a continuous Positive Airways Pressure Machine Rack according to an embodiment of the present invention.

FIG. 2 is a perspective view illustrating a back panel of the continuous positive airways pressure machine rack according to an embodiment of the present invention of FIG. 1.

FIG. 3 is a perspective view illustrating a flexible cover member of the continuous positive airways pressure machine rack according to an embodiment of the present invention of FIG. 1.

FIG. 4 is a perspective view illustrating a combination of a continuous positive airways pressure machine and a continuous positive airways pressure machine rack according to an embodiment of the present invention of FIG. 1.

FIG. 5 is an exploded view illustrating a first clamp member and second clamp member of the combination of a continuous positive airways pressure machine and a continuous positive airways pressure machine rack according to an embodiment of the present invention of FIG. 1.

FIG. 6 is another exploded view illustrating another view of the combination of a continuous positive airways pressure machine and a continuous positive airways pressure machine rack according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to an organizing device for medical equipment and more specifically relates to a continuous positive airways pressure machine rack (Entitled CPAP Storage and Organizing Unit) structured and arranged to neatly and conveniently store CPAP accessories, with easy-to-use hose clamps, mask hooks, wire management grommet holes, and accessory shelves.

Generally speaking, the CPAP Storage and Organizing Unit neatly and conveniently stores CPAP accessories, with easy-to-use hose clamps, mask hooks, wire management grommet holes, and accessory shelves. Spend less time fumbling with your CPAP and more time resting with CPAP Storage and Organizing Unit.

Easy to Use and Packed with Features and Benefits:
Hose clamps and hooks support CPAP hose and mask
Includes shelves for organizing accessories and grommet holes for wire management
Front screen cover hides contents
Keeps CPAP accessories neat and accessible, and eliminates tangles
Waterproof bottom contains spills
Helps CPAP users sleep more safely and soundly

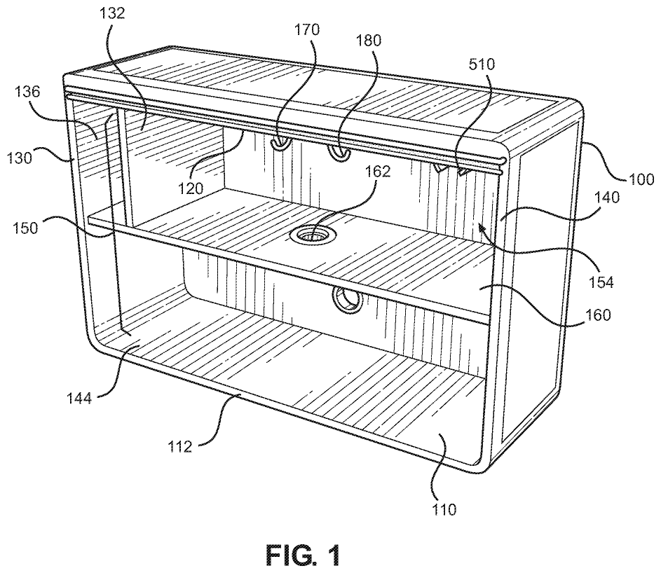

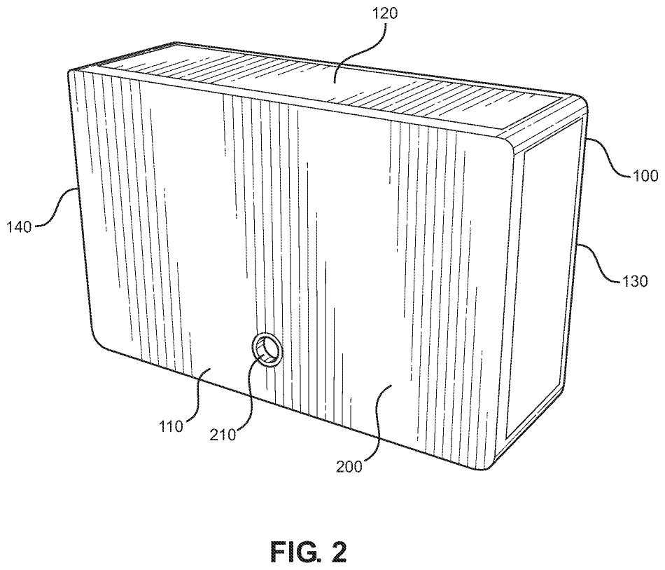

Referring now to FIGS. 1-3, showing perspective views illustrating continuous positive airways pressure machine rack 100 according to an embodiment of the present invention of FIG. 1.

Continuous positive airways pressure machine rack 100 comprising: bottom panel 110; top panel 120; back panel 200 including aperture 210; first side panel 130; second side panel 140; shelf panel 160 including aperture 162; first hook member 170; second hook member 180; and clamp member 500. Wherein top panel 120, bottom panel 110, back panel 200, and first side panel 130 and second side panel 140 form interior volume 150 and open front area 154. Wherein interior volume 150 has dimensions including 18 inches in height and between 6 and 8 inches in depth.

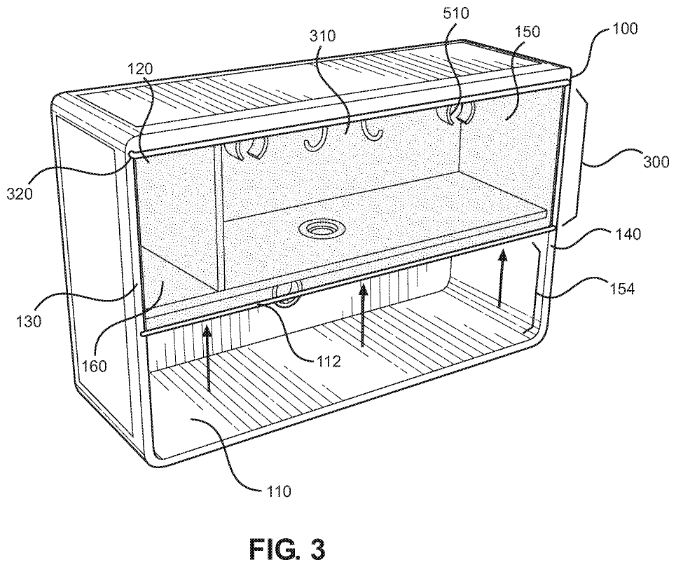

Continuous positive airways pressure machine rack 100 further comprising flexible cover member 300 attached on a side portion thereof to an edge portion of top panel member 120 and adapted to removably cover open front area 154 of continuous positive airways pressure machine rack 100 as shown in FIG. 3. Wherein flexible cover member 300 is formed as roller blind 310 having roller member 320 extending between first panel 130 and second side panel 140.

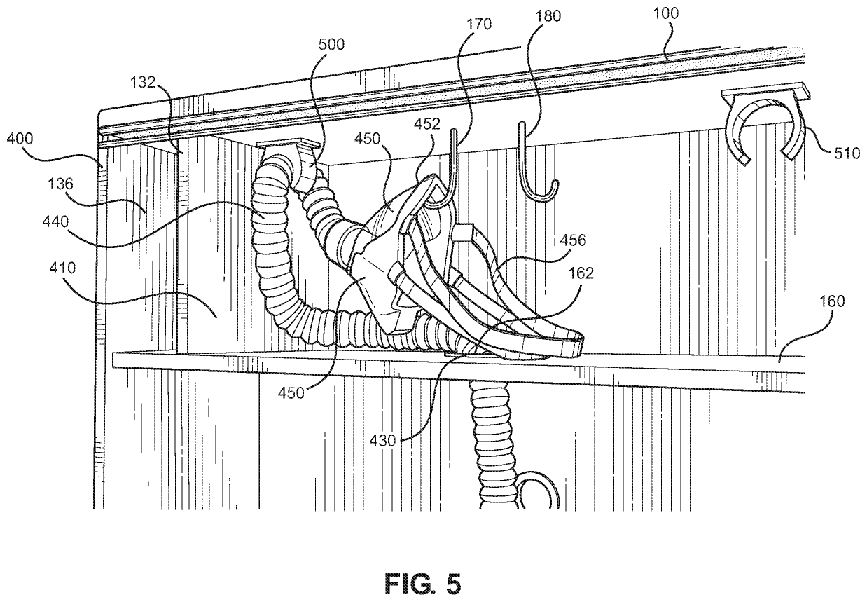

Bottom panel 110 is adapted to retain base member 420 of continuous positive airways pressure machine 410 thereon. Wherein bottom panel 110 includes lip portion 112 on an outer edge thereof adjacent open front area 154 and extending along a substantial length thereof. Wherein corner portions formed by bottom panel 110, first side panel 130 and second side panel 140, back panel 200, and lip portion 112 include waterproofing material 144 thereon. Wherein bottom panel 110 includes rubber liner 112 upon a substantial portion of an interior surface thereof.

Wherein aperture 162 of shelf panel 160 extends through a middle portion thereof and is adapted to allow electrical cords 430 of continuous positive airways pressure machine 410 to pass therethrough. Wherein back panel 200 is connected to and extends between edge portions of bottom panel 110 and top panel 120. Wherein aperture 162 extends through a middle portion thereof and is adapted to allow hose member 440 of continuous positive airways pressure machine 410 pass therethrough.

Wherein first side panel 130 is connected to and extends between edge portions of bottom panel 110 and top panel 120 at respective first end portions thereof, and is connected to and extends from the back panel 200 at a first end portion thereof. Wherein second side panel 140 is connected to and extends between edge portions of bottom panel 110 and top panel 120 at respective second end portions thereof, and is connected to and extends from back panel 200 at second end portion thereof.

Wherein shelf panel 160 is connected to and extends between respective middle portions of the first side panel 130 and second side panel 140 and is located adjacent back panel 200. Continuous positive airways pressure machine rack 100 further comprising wall panel member 132 located in proximity to first side panel 130 and is connected between a top surface of shelf panel 160 and the inner surface top panel 120, thereby forming document slot 136.

Wherein first hook member 170 is attached to an inner surface of top panel 120 and extends downwardly therefrom and is adapted to releasably retain a portion of mask member 450 of continuous positive airways pressure machine 410 thereon. Wherein second hook member 180 is attached to an inner surface of top panel 120, extends downwardly therefrom, is spaced from first hook member 170, and is adapted to releasably retain a portion of hose 440 of continuous positive airways pressure machine 410 thereon.

Wherein clamp member 500 is attached to an inner surface of top panel 120, extends downwardly therefrom, is spaced from the first hook member 170 and second hook member 180, and is adapted to releasably retain a portion of hose 440 of continuous positive airways pressure machine 410 thereon. Continuous positive airways pressure machine rack 100 further comprising second clamp member 510 attached to an inner surface of top panel 120, extends downwardly therefrom, is spaced from first hook member 170 and second hook member 180, and is adapted to releasably retain a portion of hose 440 of continuous positive airways pressure machine 410 thereon; and wherein first hook member 170 and second hook member 180 are located in between first clamp member 500 and second clamp member 510 as shown in FIG. 5.

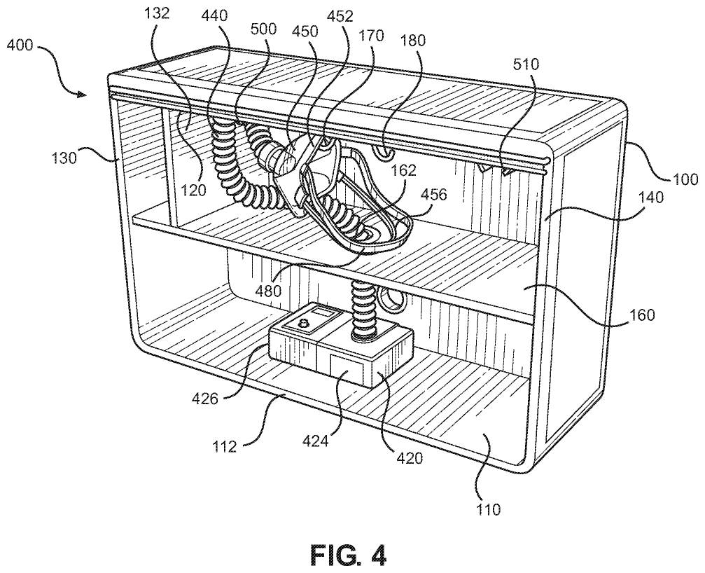

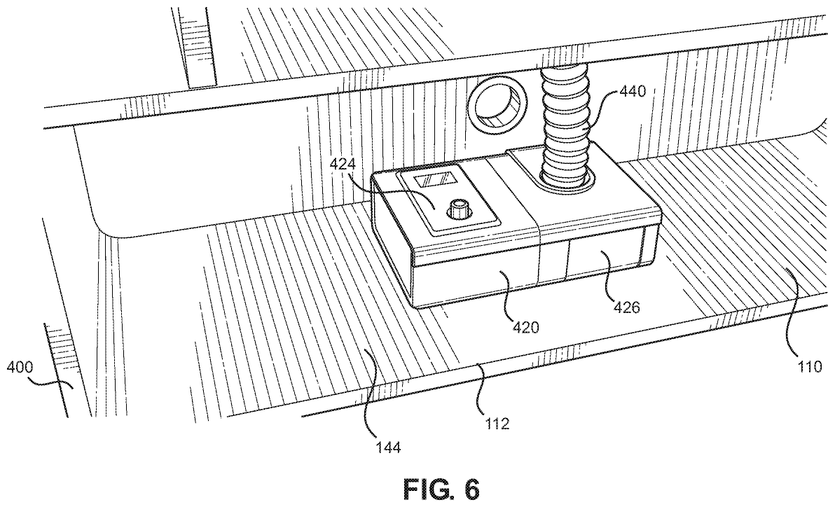

Referring now to FIGS. 4-6 are perspective views illustrating combination 400 of continuous positive airways pressure machine 410 and continuous positive airways pressure machine rack 410 according to an embodiment of the present invention of FIG. 1.

Combination 400 of continuous positive airways pressure machine 410 and continuous positive airways pressure machine rack 100, combination 400 comprising: continuous positive airways pressure machine 410 including base member 420 including motor member 424 and water reservoir 426; elongated hose member 440; and mask member 450 including face portion 452 and strap member 456. Wherein elongated hose member 440 is releasably attached to base member 420. Wherein face portion 452 is adapted to be placed on a user's face. Wherein strap member 456 is adjustably connected to face portion 452 and is adapted to extend around a user's head to releasably hold mask member 450 to the user's face.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. A continuous positive airways pressure machine rack comprising:
a bottom panel;
wherein said bottom panel is adapted to retain a base member of a continuous positive airways pressure machine thereon;
a top panel;
a back panel including:
an aperture;
wherein said aperture extends through a middle portion thereof and is adapted to allow electrical cords of said continuous positive airways pressure machine to pass therethrough;
wherein said back panel is connected to and extends between edge portions of said bottom panel and said top panel;
a first side panel;
wherein said first side panel is connected to and extends between edge portions of said bottom panel and said top panel at respective first end portions thereof, and is connected to and extends from said back panel at a first end portion thereof;
a second side panel;
wherein said second side panel is connected to and extends between edge portions of said bottom panel and said top panel at respective second end portions thereof, and is connected to and extends from said back panel at a second end portion thereof;
wherein said top panel, said bottom panel, said back panel, and said first and second side panels form an interior volume and an open front area;
a shelf panel including:
an aperture;
wherein said aperture extends through a middle portion thereof and is adapted to allow a hose member of said continuous positive airways pressure machine pass therethrough;
wherein said shelf panel is connected to and extends between respective middle portions of said first and second side panels, and is located adjacent said back panel;
a first hook member;
wherein said first hook member is attached to an inner surface of said top panel and extends downwardly therefrom and is adapted to releasably retain a portion of a mask member of said continuous positive airways pressure machine thereon;
a second hook member;
wherein said second hook member is attached to an inner surface of said top panel, extends downwardly therefrom, is spaced from said first hook member, and is adapted to releasably retain a portion of said hose of said continuous positive airways pressure machine thereon; and
a clamp member;
wherein said clamp member is attached to an inner surface of said top panel, extends downwardly therefrom, is spaced from said first and second hook members, and is adapted to releasably retain a portion of said hose of said continuous positive airways pressure machine thereon.

2. The continuous positive airways pressure machine rack of claim 1, further comprising a wall panel member located in proximity to said first side panel and is connected between a top surface of said shelf member and said inner surface of said to panel, thereby forming a document slot.

3. The continuous positive airways pressure machine rack of claim 1, further comprising a second clamp member attached to an inner surface of said top panel, extends downwardly therefrom, is spaced from said first and second hook members, and is adapted to releasably retain a portion of said hose of said continuous positive airways pressure machine thereon; and wherein said first and second hook members are located in between the first clamp member and said second clamp member.

4. The continuous positive airways pressure machine rack of claim 1, further comprising a flexible cover member attached on a side portion thereof to an edge portion of said top panel member and adapted to removably cover said open front area of said continuous positive airways pressure machine rack.

5. The continuous positive airways pressure machine rack of claim 4, wherein said flexible cover member is formed as a roller blind having a roller member extending between said first and second side panels.

6. The continuous positive airways pressure machine rack of claim 1, wherein said bottom panel includes a lip portion on an outer edge thereof adjacent said open front area and extending along a substantial length thereof.

7. The continuous positive airways pressure machine rack of claim 6, wherein corner portions formed by said bottom panel, said first and second side panels, said back panel, and said lip portion include a waterproofing material thereon.

8. The continuous positive airways pressure machine rack of claim 6, wherein said bottom panel includes a rubber liner upon a substantial portion of an interior surface thereof.

9. The continuous positive airways pressure machine rack of claim 1, wherein said interior volume has dimensions including 18 inches in height and between 6 and 8 inches in depth.

10. A combination of a continuous positive airways pressure machine and a continuous positive airways pressure machine rack, said combination comprising:
a continuous positive airways pressure machine including:
a base member including:
a motor member; and
a water reservoir;
an elongated hose member;
wherein said elongated hose member is releasably attached to said base member; and
a mask member including:
a face portion;
wherein said face portion is adapted to be placed on a user's face; and
a strap member;
wherein said strap member is adjustably connected to said face portion and is adapted to extend around a user's head to releasably hold said mask member to said user's face; and
a continuous positive airways pressure machine rack comprising:
a bottom panel;
wherein said bottom panel is adapted to retain a base member of a continuous positive airways pressure machine thereon;
a top panel;
a back panel including:
an aperture;
wherein said aperture extends through a middle portion thereof and is adapted to allow electrical cords of said continuous positive airways pressure machine to pass therethrough;
wherein said back panel is connected to and extends between edge portions of said bottom panel and said top panel;
a first side panel;
wherein said first side panel is connected to and extends between edge portions of said bottom panel and said top panel at respective first end portions thereof, and is connected to and extends from said back panel at a first end portion thereof;
a second side panel;
wherein said second side panel is connected to and extends between edge portions of said bottom panel and said top panel at respective second end portions thereof, and is connected to and extends from said back panel at a second end portion thereof;
wherein said top panel, said bottom panel, said back panel, and said first and second side panels form an interior volume and an open front area;
a shelf panel including:
an aperture;
wherein said aperture extends through a middle portion thereof and is adapted to allow a hose member of said continuous positive airways pressure machine pass therethrough;
wherein said shelf panel is connected to and extends between respective middle portions of said first and second side panels, and is located adjacent said back panel;
a first hook member;
wherein said first hook member is attached to an inner surface of said top panel and extends downwardly therefrom and is adapted to releasably retain a portion of a mask member of said continuous positive airways pressure machine thereon;
a second hook member;
wherein said second hook member is attached to an inner surface of said top panel, extends downwardly therefrom, is spaced from said first hook member, and is adapted to releasably retain a portion of said hose of said continuous positive airways pressure machine thereon; and
a clamp member;
wherein said clamp member is attached to an inner surface of said top panel, extends downwardly therefrom, is spaced from said first and second hook members, and is adapted to releasably retain a portion of said hose of said continuous positive airways pressure machine thereon.

11. The combination of claim 10, wherein said continuous positive airways pressure machine rack further comprises a wall panel member located in proximity to said first side panel and is connected between a top surface of said shelf member and said inner surface of said to panel, thereby forming a document slot.

12. The combination of claim 10, wherein said continuous positive airways pressure machine rack further comprises a second clamp member attached to an inner surface of said top panel, extends downwardly therefrom, is spaced from said first and second hook members, and is adapted to releasably retain a portion of said hose of said continuous positive airways pressure machine thereon; and wherein said first and second hook members are located in between the first clamp member and said second clamp member.

13. The combination of claim 10, wherein said continuous positive airways pressure machine rack further comprises a flexible cover member attached on a side portion thereof to an edge portion of said top panel member and adapted to removably cover said open front area of said continuous positive airways pressure machine rack.

14. The combination of claim 13, wherein said flexible cover member is formed as a roller blind having a roller member extending between said first and second side panels.

15. The combination of claim 10, wherein said bottom panel of said continuous positive airways pressure machine rack further comprises includes a lip portion on an outer edge thereof adjacent said open front area and extending along a substantial length thereof.

16. The combination of claim 15, wherein corner portions formed by said bottom panel, said first and second side panels, said back panel, and said lip portion include a waterproofing material thereon.

17. The combination of claim 15, wherein said bottom panel includes a rubber liner upon a substantial portion of an interior surface thereof.

18. The combination of claim 10, wherein said interior volume of said continuous positive airways pressure machine rack has dimensions including 18 inches in height and between 6 and 8 inches in depth.
